(19) United States
(12) Patent Application Publication
(10) Pub. No.: US 2025/0009544 A1
(43) Pub. Date: Jan. 9, 2025

(54) Knob adjustable back orthopedic belt

(71) Applicant: Dongguan Fushun Sports Goods Co., Ltd., Dongguan (CN)

(72) Inventor: Xiaogao Gu, Ji'an (CN)

(21) Appl. No.: 18/358,129

(22) Filed: Jul. 25, 2023

(30) Foreign Application Priority Data
Jul. 5, 2023 (CN) 2023200890258

(51) Int. Cl. A61F 5/02

(52) U.S. Cl. CPC A61F 5/026

(57) ABSTRACT

A knob adjustable back orthopedic belt is disclosed, including an orthopedic belt body. The orthopedic belt includes a spinal fixation part, waist belts and shoulder straps. The spinal fixation part at its top is provided with a cross bib, the cross bib is provided with four sets of perforations, the waist belts are provided with knobs near a portion of the spinal fixation part, and the knobs are wrapped with string. A fixing band is provided at a vertical center of the spinal fixation part, both sides of the fixing band is configured to connect to elastic bands, and the elastic bands are configured to be connected at their ends to the string. The knob adjustable back orthopedic belt can be effectively adapted to needs of patients with different body shapes and has stronger universality.

### TECHNICAL FIELD

The present application belongs to the technical field of back orthopedic belt, and specifically relates to a new knob adjustable back orthopedic belt.

### BACKGROUND

Hunchback is a relatively common spinal deformation, is caused by thoracic spine back protrusion of morphological changes, mostly seen in old age spinal deformation, sitting and standing incorrect posture or rickets and ankylosing spondylitis and other diseases. Hunchback according to whether may be corrected is divided into movable hunchback and fixed hunchback two kinds, and in order to correct phenomenons of young people's hunchback, thus people need to develop a kind of hunchback corrective belt.

Existing hunchback orthopedic belts are usually fixed in size, for example, CN 206063272 U discloses a hunchback orthopedic belt, which constrain a patient's back by means of two elastic support strips, and the support strips themselves have a certain degree of elasticity. As a result, patient's movement is not excessively hindered. However, the hunchback orthopedic belt also has a very obvious defect, that is, a height of the patient must meet requirements of the support strips, and adolescents are generally in the period of high-speed development of the body, and their height grows very quickly, therefore, the orthopedic belt is prone to a problem that the corrective effect of the orthopedic belt is reduced due to the development of the patient's height. Therefore, we propose a new knob adjustable back orthopedic belt to solve above mentioned drawbacks.

### SUMMARY

The present disclosure provides a new knob adjustable back orthopedic belt that can be effectively adapted to needs of patients with different body shapes and has stronger universality to address above problems.

Specifically the technical solutions of the present disclosure are as follows. In some embodiments, the present disclosure provides a new knob adjustable back orthopedic belt, including an orthopedic belt body. The orthopedic belt body includes a spinal fixation part, waist belts and shoulder straps. The waist belts are provided in two sets and are configured to be integrally connected to top of left and right sides of the spinal fixation part, respectively. The shoulder straps are provided in two sets and are configured to be integrally attached to top of left and right sides of the spinal fixation part, respectively. The spinal fixation part at it top is provided with a cross bib, the cross bib is provided with four sets of perforations, the waist belts are provided with knobs near a portion of the spinal fixation part, and the knobs are wrapped with string. A fixing band is provided at a vertical center of the spinal fixation part, both sides of the fixing band is configured to connect to elastic bands, and the elastic bands are configured to be connected at their ends to the string.

Preferably, in some embodiments, the waist belts are configured with Velcro I.

Preferably, in some embodiments, the shoulder straps at ends are configured with Velcro II.

Preferably, in some embodiments, the shoulder straps are configured to be cross threaded within the perforations of the cross bib.

Preferably, in some embodiments, the cross bib is configured as a diamond-shaped structure.

The knob adjustable back orthopedic belt of the present disclosure at least has following technical advantages. The knob adjustable back orthopedic belt are configured with knobs, string and elastic bands, the knobs can be turned to tighten by pressing them down, the elastic bands can be tightened by tightening the bands, pulling the knobs away can loosen the elastic bands so as to conveniently adjust the degree of lumbar looseness, which can effectively adapt to the needs of patients with different body shapes, and universality is more powerful. In addition, use of cross bib to limit the position of the shoulder straps is conducive to the shoulder straps to fully open the patient's shoulders and improve the corrective effect.

### BRIEF DESCRIPTION OF THE DRAWINGS

FIG. 1 shows a schematic diagram of an overall structure of a new knob adjustable back orthopedic belt in accordance with some embodiments of the present disclosure.

FIG. 2 shows a partially enlarged view of FIG. 1 at A.

FIG. 3 shows a schematic diagram of the knob adjustable back orthopedic belt in use in accordance with some embodiments of the present disclosure.

In the drawings, the reference signs are as follows. 1. orthopedic belt body, 2. spinal fixation part, 3. waist belt, 4. shoulder strap, 5. cross bib, 6. knob, 7. string, 8. fixing band, 9. elastic band, 10. Velcro I, 11. Velcro II.

### DETAILED DESCRIPTION OF THE EMBODIMENTS

The technical solutions of the present disclosure will be clearly and completely described below in conjunction with the accompanying drawings, and it is clear that the described embodiments are a part of the embodiments of the present disclosure, and not all of them. Based on the embodiments in the present disclosure, all other embodiments obtained by a person of ordinary sill in the art without making creative labor fall within the scope of protection of the present disclosure.

In the description of the present disclosure, it should be noted that the terms “center”, “top”, “bottom”, “left”, “right”, “vertical”, “horizontal”, “inner”, “outer”, etc. indicating the orientation or positional relationships are based on the orientation or positional relationships shown in the accompanying drawings, and are intended only to facilitate and simplify the description of the present disclosure, not to indicate or imply that the device or element referred to must have a particular orientation, be constructed and operate in a particular orientation, and therefore are not to be construed as limiting the present disclosure. Furthermore, the terms “first”, “second”, and “third” are used for descriptive purposes only and are not to be construed as indicating or implying relative importance. The present disclosure is described in further detail below in conjunction with the accompanying drawings.

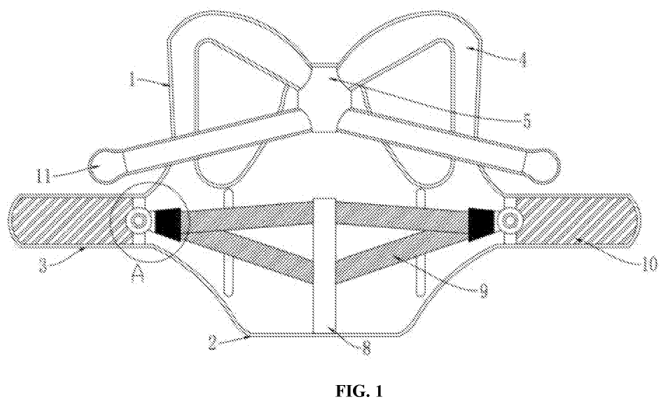

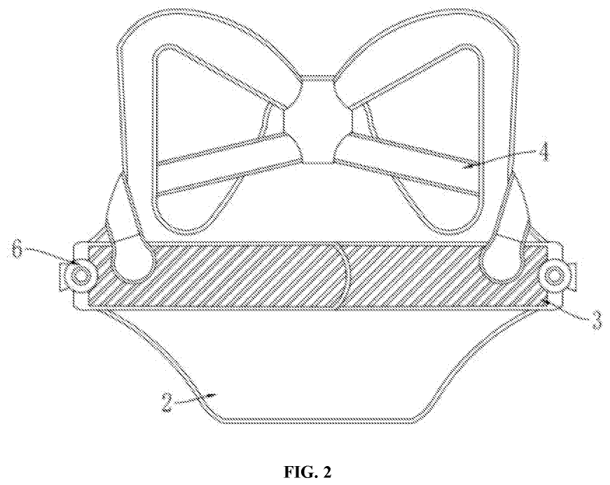

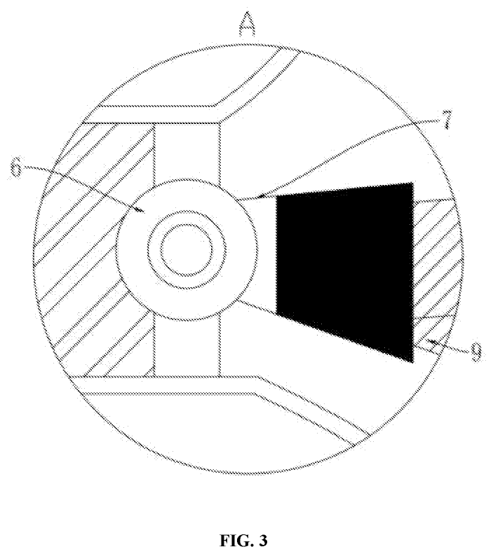

As shown in FIG. 1 to FIG. 3, in some embodiments, the present disclosure provides a new knob adjustable back orthopedic belt, including an orthopedic belt body 1. The orthopedic belt body 1 includes a spinal fixation part 2, waist belts 3 and shoulder straps 4. The waist belts 3 are provided in two sets and are configured to be integrally connected to top of left and right sides of the spinal fixation part 2, respectively. The shoulder straps 4 are provided in two sets and are configured to be integrally attached to top of left and right sides of the spinal fixation part 2, respectively. The spinal fixation part 2 at its top is provided with a cross bib 5, the cross bib 5 is provided with four sets of perforations, the waist belts 3 are provided with knobs 6 near a portion of the spinal fixation part 2, and the knobs 6 are wrapped with string 7. A fixing band 8 is provided at a vertical center of the spinal fixation part 2, both sides of the fixing band 8 is configured to connect to elastic bands 9, and the elastic bands 9 are configured to be connected at their ends to the string 7. By such designs, the knobs 6 can be turned to tighten by pressing them down, the elastic bands 9 can be tightened by tightening the bands. Pulling the knobs 6 away can loosen the elastic bands so as to conveniently adjust the degree of lumbar looseness, which can effectively adapt to the needs of patients with different body shapes, and universality is more powerful.

In some embodiments, the waist belts are configured with Velcro I 10.

In some embodiments, the shoulder straps 4 are configured to be cross threaded within the perforations of the cross bib 5, and the shoulder straps (4) at ends are configured with Velcro II 11. To use, a middle part of the shoulder straps 4 is wrapped around the patient's shoulder while the end of the shoulder straps 4 is wrapped around the patient's chest and attached to the waist belts by Velcro II 11.

In specific use, the shoulder straps 4 through the patient's two shoulders respectively, while a side of the spinal fixation part 2 configured with the cross bib 5 fit on the patient's back, according to the patient's body type needs to be adjusted to the lumbar looseness, the knobs 6 are pressed down and then can be turned to tighten, and the elastic bands 9 are tightened by threading the bands, the knobs 6 are pulled away, the elastic bands are loosen. And after adjusting them to a proper looseness, the two sets of waist belts 3 are wrapped around the two sides of the lumbar, and meanwhile, a middle part of the shoulder straps 4 is wrapped around the patient's shoulder while the ends of the shoulder straps 4 are then wrapped around the patient's chest and attached to the Velcro I 10 on the waist belts 3 via the Velcro II 11.

The above description of the present disclosure and its embodiments is not limiting, and what is shown in the accompanying drawing is only one of the embodiments of the present disclosure, and an actual structure is not limited to it. All in all if a person of ordinary skill in the art is inspired by it, without departing from the creation purpose of the present disclosure, and designing without creatively a structural way and embodiment similar to this technical solution, all shall fall within the scope of protection of the application.

What is claimed is:

1. A knob adjustable back orthopedic belt, comprising an orthopedic belt body (1), wherein
the orthopedic belt body (1) comprises a spinal fixation part (2), waist belts (3) and shoulder straps (4);
the waist belts (3) are provided in two sets and are configured to be integrally connected to top of left and right sides of the spinal fixation part (2), respectively;
the shoulder straps (4) are provided in two sets and are configured to be integrally attached to top of left and right sides of the spinal fixation part (2), respectively;
the spinal fixation part (2) at its top is provided with a cross bib (5), the cross bib (5) is provided with four sets of perforations, the waist belts (3) are provided with knobs (6) near a portion of the spinal fixation part (2), and the knobs (6) are wrapped with string (7); and
a fixing band (8) is provided at a vertical center of the spinal fixation part (2), both sides of the fixing band (8) is configured to connect to elastic bands (9), and the elastic bands (9) are configured to be connected at their ends to the string (7).

2. The knob adjustable back orthopedic belt according to claim 1, wherein the waist belts are configured with Velcro I (10).

3. The knob adjustable back orthopedic belt according to claim 2, wherein the shoulder straps (4) at ends are configured with Velcro II (11).

4. The knob adjustable back orthopedic belt according to claim 3, wherein the shoulder straps (4) are configured to be cross threaded within the perforations of the cross bib (5).

5. The knob adjustable back orthopedic belt according to claim 4, wherein the cross bib (5) is configured as a diamond-shaped structure.
